(19) United States
(12) Patent Application Publication
(10) Pub. No.: US 2020/0390465 A1
(43) Pub. Date: Dec. 17, 2020

(54) GUIDE DEVICE FOR BIOPSY NEEDLE

(71) Applicant: Otto-von-Guericke-Universität Magdeburg, Magdeburg (DE)

(72) Inventor: Juan Sebastián SÁNCHEZ LÓPEZ, Magdeburg (DE)

(73) Assignee: Otto-von-Guericke-Universität Magdeburg, Magdeburg (DE)

(21) Appl. No.: 16/766,683

(22) PCT Filed: Nov. 22, 2018
(86) PCT No.: PCT/EP2018/082235

§ 371 Date: May 24, 2020

(30) Foreign Application Priority Data
Nov. 24, 2017 (DE) 10 2017 127 871.0
Mar. 16, 2018 (DE) 10 2018 106 198.6

(51) Int. Cl. A61B 17/34; A61B 10/02; A61B 90/00

(52) U.S. Cl. CPC A61B 17/3403; A61B 10/0233; A61B 2090/3966; A61B 2017/3407; A61B 2017/3409; A61B 90/39

(57) ABSTRACT

The invention relates to a guide device (1) for guiding and positioning a biopsy needle (100) at a puncture site (101) on the body of a patient, wherein the guide device (1) has a base (2) and a needle holder (3), on which the biopsy needle (100) is guided along the longitudinal extent of same, wherein the position of the needle holder (3) relative to the base (2) can be changed. The invention proposes that the position of the needle holder (3) relative to the base (2) can be changed in only two degrees of freedom (4, 5), wherein the biopsy needle (100) guided on the needle holder (3) always punctures the same puncture site (101) when the position of the needle holder (3) relative to the base (2) changes.

The invention relates to a guide device for guiding and positioning a biopsy needle at a puncture site on the body of a patient, wherein the guide device has a base and a needle holder, on which the biopsy needle is guided along the longitudinal extent of same, wherein the position of the needle holder relative to the base can be changed.

The capabilities of image-guided biopsy procedures have increased significantly in the last ten years. In 2015, more than 14 million biopsy procedures were recorded in Germany alone. Moreover, the number of new cases will double by 2030 because of the global increase in cancers.

Biopsies are generally performed manually with imaging by ultrasound, computed tomography or magnetic resonance imaging. The use of the individual imaging methods depends on the organ to be punctured, the accuracy required, the costs and the surgeon's specialization. In all three cases of imaging guidance, a needle holder is required to facilitate the procedure and to maintain the alignment of the biopsy needle during the procedure. Otherwise the surgeon would have to guide the biopsy needle manually, leading to inaccurate results. In the case of X-ray imaging, this is even harmful for the surgeon.

From US 2012/0022368 A1 a guide device is known with which the biopsy needle can be introduced vertically and laterally. However, the system has a limited range of movement and requires the fixation of three screws, which is inconvenient for the surgeon.

In general, there is a need for instruments for biopsy assistance to increase accuracy, accelerate the biopsy workflow and provide greater convenience for the surgeon.

It is therefore the object of the present invention to provide a guide device for guiding and positioning a biopsy needle at a puncture site on the body of a patient, which is easy to operate and permits a targeted positioning of the biopsy needle.

This object is achieved by a guide device having the features of claim 1.

Owing to the fact that the position of the needle holder relative to the base can only be changed in two degrees of freedom, with the biopsy needle that is guided on or in the needle holder always puncturing the same puncture site when the position of the needle holder relative to the base is changed, simple operation is possible based on the two degrees of freedom. Because the biopsy needle in the needle holder is aimed at the same puncture site, the same entry point can always be used for puncturing with the biopsy needle with a single alignment of the guide device, permitting a targeted positioning of the biopsy needle. The guide device according to the invention brings advantages over other products on the market and improves the way in which biopsy procedures are performed in combination with all imaging methods.

Advantageous embodiments and developments of the invention can be taken from the dependent claims. It should be pointed out that the individual features listed in the claims can also be combined in any technologically meaningful way, thus demonstrating further embodiments of the invention.

According to an advantageous embodiment of the invention it is provided that one degree of freedom is formed by a pivot axis, wherein the puncture site of the biopsy needle being guided on the needle holder lies on the pivot axis. The change in the position of the needle holder relative to the base thus permits an accurate positioning of the biopsy needle being held in the needle holder. Owing to the fact that the pivot axis formed for the first degree of freedom meets the puncture site of a biopsy needle being held in the needle holder in an imaginary extension in the axis direction, the same puncture site selected by the surgeon can always be used for the puncture when the needle holder is aligned over the first degree of freedom. By changing the position of the needle holder, therefore, the injection angle of the biopsy needle being guided in the needle holder can be changed very simply without a resulting alteration to the puncture site that is targeted by the biopsy needle being held in the needle holder.

Particularly advantageous is an embodiment that provides that the other degree of freedom is formed by a guide curve that is at least in part circular, wherein the center point of the circle formed by the guide curve lies at the puncture site. In this way, the injection angle of the biopsy needle being held in the needle holder can also be changed very simply via the second degree of freedom without a resulting alteration to the position of the puncture site on the patient's body that is targeted by the biopsy needle being held in the needle holder.

A particularly advantageous version of the invention provides that the guide curve is configured so as to be pivotable relative to the base around the pivot axis, said pivot axis lying in the circular plane of the guide curve. Particularly by combining the two degrees of freedom formed by the pivot axis and the guide curve, a guide device is obtained that is particularly easy to handle. The injection angle can be varied in a polar and an azimuthal direction without the position of the puncture site altering. The intuitive operation of the guide device accelerates the biopsy workflow and offers the surgeon greater convenience.

An advantageous version provides that the guide curve has at least one position scale for the sliding of the needle holder on the guide curve. With a position scale that offers reference points for the sliding of the needle holder on the guide curve, the surgeon is better able to plan the desired injection angle at the puncture site during the biopsy and to define it reproducibly using the guide device. The position scale on the guide curve should preferably offer specifications for the angular position over the second degree of freedom that can be adjusted by sliding the needle holder on the guide curve. Angle specifications of 0° to 45° and to −45° should offer the surgeon adequate assistance in selecting the injection angle.

Particularly advantageous is an embodiment in which it is provided that a position scale is also provided for the pivoting of the needle holder relative to the base around the pivot axis. The position scale should preferably offer specifications for the angular position of the guide curve relative to the base with reference to the first degree of freedom that can be adjusted. Here, angle specifications of 0° to 45° and to −45° should offer the surgeon adequate assistance in selecting the injection angle.

A particularly advantageous embodiment of the invention provides that the position of the needle holder relative to the base is lockable with only two locking elements. With the locking of the position of the needle holder by way of just two locking elements, a simple and rapid operation of the guide device can be achieved, and so the surgeon can intuitively define and rapidly secure the position of the needle holder during the biopsy.

It is of particular advantage according to an embodiment that the position of the needle holder can be changed in at least one degree of freedom by way of at least one remotely controllable actuator. A particularly advantageous version provides that the position of the needle holder can be changed in two degrees of freedom by way of two remotely controllable actuators. With a remotely controllable actuator, the position of the needle holder can be changed very simply and precisely from a distance. This offers a particular advantage in confined spaces, e.g. in an MRI facility or during an operation with X-ray imaging.

Particularly advantageous is an embodiment in which it is provided that the guide device has at least one marker. With a marker on the guide device, the precise position of the guide device can be determined particularly easily in an imaging method. The marker helps to position and align the guide device at the puncture site easily using imaging such as X-ray, ultrasound or MRI. The marker makes the position and alignment of the guide device visible on the image that is generated.

Further features, details and advantages of the invention can be obtained on the basis of the following description and with reference to the drawings. Exemplary embodiments of the invention are illustrated in the following drawings and will be described in more detail below. Objects corresponding to one another are provided with the same reference characters in all the figures. The figures show the following:

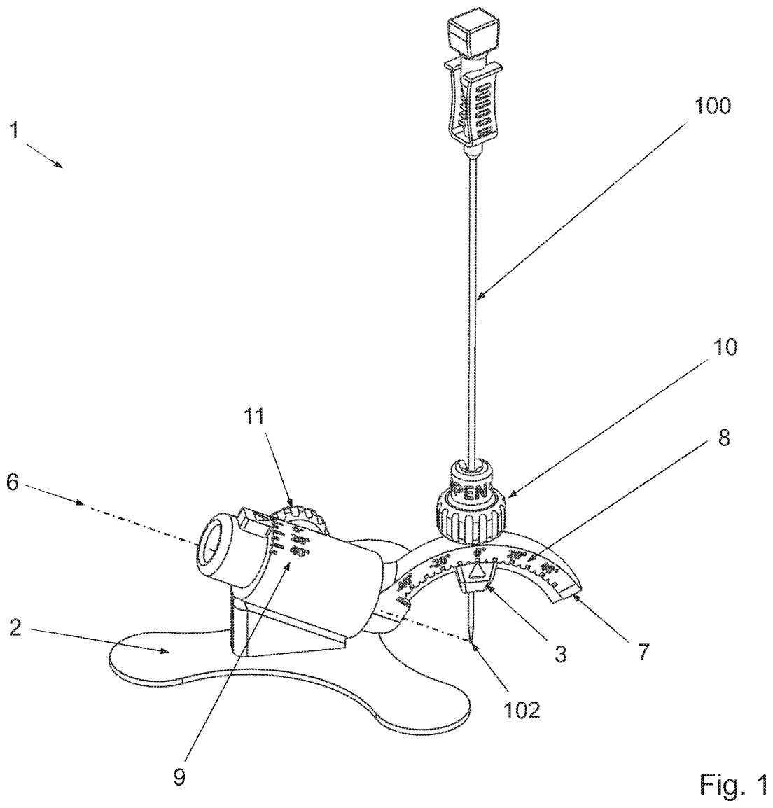

FIG. 1: guide device according to the invention with a biopsy needle,

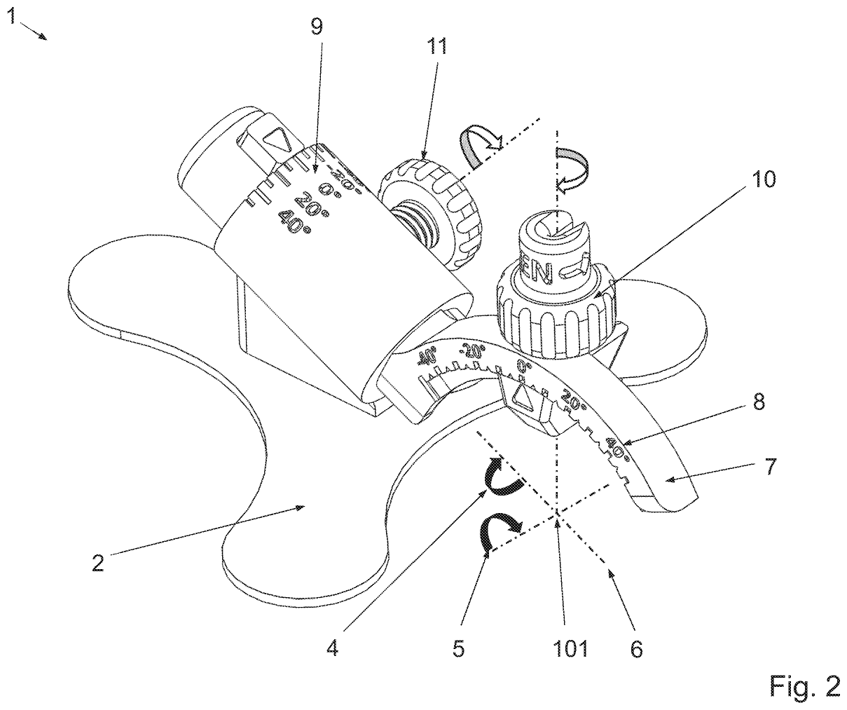

FIG. 2: guide device at puncture site,

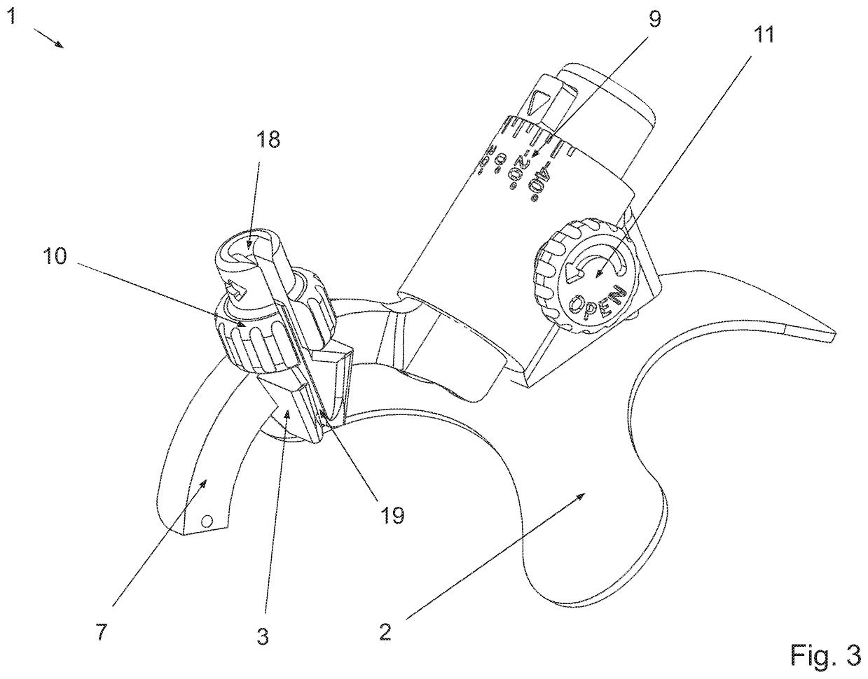

FIG. 3: disengaged guide device,

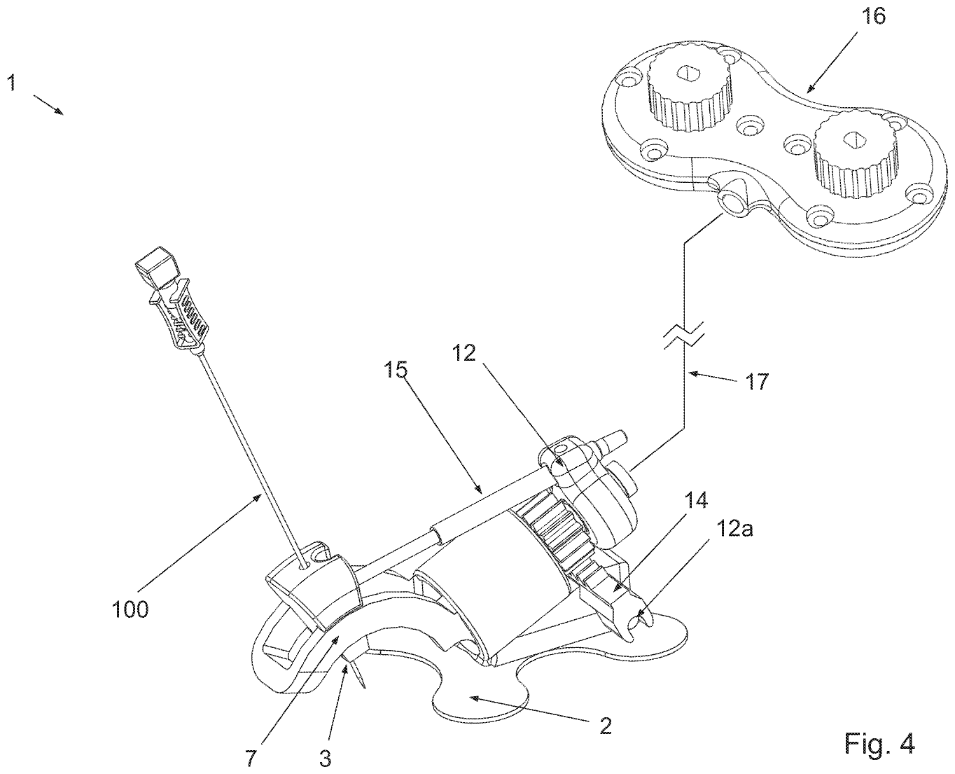

FIG. 4: guide device with actuator,

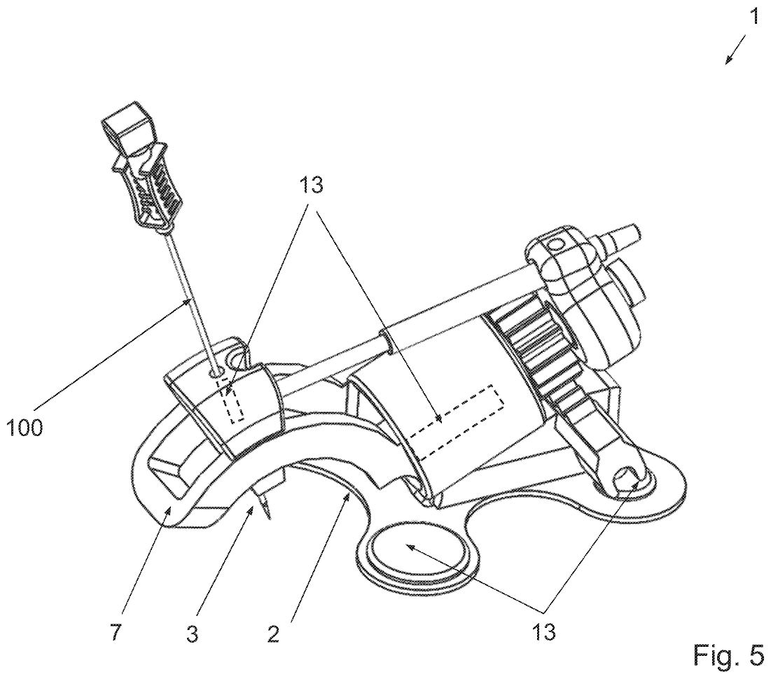

FIG. 5: guide device with markers.

A guide device according to the invention is shown in the figures labelled with the reference character 1. The guide device 1 has a base 2, with which the guide device 1 is arranged on the body of a patient, for example. The base 2 is formed such that the guide device 1 takes up the smallest possible area on the patient. In order to arrange the guide device 1 securely on the patient's skin, a paper cover on the base 2 is removed, an adhesive being located under the paper cover. This permits a simple attachment to the patient. The support surface of the base 2 consists of a soft material, which conforms to complex anatomical shapes of the patient. This conformability of the base 2 can also be seen in FIG. 3, where the guide device 1 is shown away from the patient's body. The guide device 1 has a needle holder 3, which is configured to hold a biopsy needle 100 and guide it towards the puncture site 101 (FIG. 2) on the patient's body. The position of the needle holder 3 on the guide device 1 can be changed relative to the base 2 in only two degrees of freedom 4, 5 (FIG. 2). The injection angle of the biopsy needle 100 that is held in the needle holder 3 can be changed over these two degrees of freedom 4, 5 (FIG. 2) without changing the position of the puncture site 101 (FIG. 2). The surgeon can define the puncture site 101 (FIG. 2) of the biopsy needle 100 that is held in the needle holder 3 by positioning the base 2 on the patient's body. The injection angle can then be changed easily by changing the position of the needle holder 3 over two degrees of freedom 4, 5 (FIG. 2). The first degree of freedom 4 (FIG. 2) with which the position of the needle holder 3 relative to the base 2 can be changed is formed by a pivot axis 6, with the puncture site 101 (FIG. 2) of the biopsy needle 100 that is held in the needle holder 3 lying on the pivot axis 6. This can be seen clearly in FIG. 1, where the extension of the pivot axis 6 indicated as a dot-dash line meets the puncture site 101 marked by the biopsy needle tip 102. The second degree of freedom 5 with which the position of the needle holder 3 relative to the base 2 can be changed is formed by a guide curve 7 that is in part curved in a circular shape. The center point of the circle formed by the guide curve 7 lies at the puncture site 101 (FIG. 2). The guide curve 7, on which the needle holder 3 is guided around the puncture site 101 in a circular arc, is configured such that it can be pivoted relative to the base 2 by the pivot axis 6 that is formed. The pivot axis 6 lies in the circular plane of the guide curve 7. The pivoting movement (first degree of freedom 4) is indicated in FIG. 2 as a curved arrow around the pivot axis 6. On the guide curve 7, the needle holder 3 is guided in a circular arc shape around the puncture site 101, giving the second degree of freedom 5 in the positioning of the needle holder 3. This second degree of freedom 5 is indicated in FIG. 2 by a rotating movement around the axis passing through the center point of the circle (puncture site 101) shown as an arrow. In order to be able to define a precise and reproducible setting of the injection angle of the biopsy needle 100 that is held in the needle holder 3, the guide curve 7 has an angle scale 8 going from −45° to +45°. This permits the position of the needle holder 3 on the guide curve 7 to be selected and modified very easily. The needle holder 3 can be locked on the guide curve 7 with a first locking element 10. For this purpose, the locking element 10 in the form of a screw nut has to be tightened with approximately a half turn. This enables the position of the needle holder 3 on the guide curve 7 to be defined easily. The position of the guide curve 7 relative to the base 2 can be locked by a second locking element 11. In order to define the position of the guide curve 7 relative to the base 2, a position scale 9 is likewise provided for the pivoting of the needle holder 3 relative to the base 2 in the pivot axis 6. This scale 9 can also have angle specifications of between −45° and +45°.

The guide device 1 preferably consists of multiple plastic components that are suitable for use in all possible imaging methods. The guide device 1 enables the surgeon to position and align the biopsy needle 100 precisely for planning and needle guiding in biopsies or other percutaneous procedures.

It can be seen clearly in FIG. 2 that the guide device 1 has only two degrees of freedom 4, 5 for needle alignment after a puncture site 101 has been defined for the biopsy. Different angular positions of the biopsy needle 100 (FIG. 1) relative to the patient's body can be selected via the two degrees of freedom 4, 5, with the biopsy needle 100 (FIG. 1) always being aimed at the same puncture site 101. The degrees of freedom 4, 5 are defined on the one hand by a circular guide curve 7 for the needle holder 3 and on the other hand by a pivot axis 6, with which the guide curve 7 can be pivoted relative to the base 2. The needle holder 3 is guided around the puncture site 101 on the circular guide curve 7. With the locking elements 10, 11 in the form of screw nuts, the selected locations of the degrees of freedom 4, 5 can be fixed, so that the position of the needle holder 3 relative to the base 2 is locked. By turning two ergonomic nuts 10, 11, the system is thus completely locked in the desired alignment of the biopsy needle 100 (FIG. 1). Two position scales 8, 9 with angles of 0° to 45° and −45° are located on the guide device 1 to assist the surgeon with alignment. The position scales 8, 9 have angle specifications in both directions to assist the surgeon with needle alignment.

In addition, the guide device 1 is designed such that the surgeon can monitor the biopsy needle 100 (FIG. 1) while it is being introduced and has free access. To assist in biopsies and other percutaneous procedures, the guide device 1 enables the needle 100 (FIG. 1) to be inserted both vertically via the vertical needle access 18 and laterally via the lateral needle access 19, as can be seen from FIG. 3. For the vertical insertion of the biopsy needle 100 (FIG. 1), the needle holder 3 is adjusted to the desired angle and the biopsy needle 100 (FIG. 1) can be introduced into the fixed needle holder 3 by way of the vertical needle access 18. In certain procedures, the biopsy needle 100 (FIG. 1) is first placed lightly on the patient and then the guide device 1 is positioned. A vertical insertion of the needle 100 (FIG. 1) is therefore not possible. For these procedures, the guide device 1 permits the lateral placing of the needle 100 (FIG. 1) by way of the lateral needle access 19. After a half turn of the nut of the locking element 10, the biopsy needle 100 (FIG. 1) is guided and the needle holder 3 is fixed at the desired location. Particular advantages of the invention are, moreover, that the guide device 1 can also be single-use owing to the low manufacturing costs, which means that sterilization after use is unnecessary. The alignment of the biopsy needle 100 (FIG. 1) is, furthermore, robust and stable.

A further embodiment of the guide device 1 is shown in FIGS. 4 and 5. This version further improves convenience, precision and safety during the biopsy. There is extremely limited space available inside an MRI tunnel, which impedes the surgeon's freedom of movement during procedures. Furthermore, when the patient is in the MRI, the surgeon cannot change the alignment of the instrument without removing the patient. Owing to these difficulties, it is proposed that the guide device 1 be provided with two remotely controllable actuators 12, 12a, which change the position of the needle holder 3 in at least one degree of freedom 4, 5 (FIG. 2). This makes it possible for the surgeon to control the angular position of the biopsy needle 100 remotely. The actuators 12, 12a are formed by two hydraulic drivers that are connected to the needle holder 3 and the guide curve 7. This enables the angular position of the biopsy needle 100 to be changed by the actuators 12, 12a. To change the alignment of the needle holder 3, the surgeon uses a console 16, which is connected to the actuators 12, 12a via tubes 17 or another connection.

To improve accuracy and accelerate the biopsy, MRI and/or CT markers 13 are provided, with which the position of the guide device 1 can be determined on an image recorded by the MRI or CT instrument. The preferred positions of the markers 13 on the guide device 1 are indicated in FIG. 5. As well as the position of the guide device 1, the angle of the needle holder 3 and the location of the guide curve 7 can also be established with the aid of the markers 13. The markers 13 preferably contain X-ray-opaque material for visualizing the guide device 1 by X-ray images.

### LIST OF REFERENCE CHARACTERS

1 Guide device
2 Base
3 Needle holder
4 First degree of freedom
5 Second degree of freedom
6 Pivot axis
7 Guide curve
8 First position scale
9 Second position scale
10 First Locking element
11 Second locking element
12 First actuator, 12a Second actuator
13 Marker
14 First driver
15 Second driver
16 Console
17 Tubes
18 Vertical needle access
19 Lateral needle access
100 Biopsy needle
101 Puncture site
102 Needle tip

1. A guide device (1) for guiding and positioning a biopsy needle (100) at a puncture site (101) on the body of a patient, wherein the guide device (1) comprises a base (2) and a needle holder (3), on which the biopsy needle (100) is guided along the longitudinal extent of same, wherein the position of the needle holder (3) relative to the base (2) can be changed,
characterized in that
the position of the needle holder (3) relative to the base (2) can be changed only in two degrees of freedom (4, 5), wherein the biopsy needle (100) being guided on the needle holder (3) always punctures the same puncture site (101) when the position of the needle holder (3) relative to the base (2) is changed.

2. The guide device (1) according to claim 1, characterized in that one degree of freedom (4) is formed by a pivot axis (6), wherein the puncture site (101) of the biopsy needle (100) being guided on the needle holder (3) lies on the pivot axis (6).

3. The guide device (1) according to claim 2, characterized in that the other degree of freedom (5) is formed by a guide curve (7) that is at least in part circular, on which the needle holder (3) is guided in a slidable manner, wherein the center point of the circle formed by the guide curve (7) lies at the puncture site (101).

4. The guide device (1) according to claim 3, characterized in that the guide curve (7) is configured such that it is pivotable around the pivot axis (6) relative to the base (2), wherein the pivot axis (6) lies in the circular plane of the guide curve (7).

5. The guide device (1) according to claim 3, characterized in that the needle holder (3) is guided on the guide curve (7) in a circular manner around the puncture site (101) of a biopsy needle (100) that is held in the needle holder (3).

6. The guide device (1) according to claim 3, characterized in that the guide curve (7) comprises at least one position scale (8) for the sliding of the needle holder on the guide curve (7).

7. The guide device (1) according to claim 2, characterized in that a position scale (9) is provided in the pivot axis (6) for the pivoting of the needle holder (3) relative to the base (2).

8. The guide device (1) according to claim 1, characterized in that the position of the needle holder (3) relative to the base (2) can be locked with just two locking elements (10, 11).

9. The guide device (1) according to claim 1, characterized in that the position of the needle holder (3) can be changed in at least one degree of freedom (4, 5) by way of at least one remotely controllable actuator (12).

10. The guide device (1) according to claim 1, characterized in that the guide device (1) comprises at least one marker (13).
